(19) United States
(12) Patent Application Publication
(10) Pub. No.: US 2008/0214920 A1
(43) Pub. Date: Sep. 4, 2008

(54) Apparatus and Method For Coupling Implanted Electrodes to Nervous Tissue

(75) Inventors: Matthias Merz, Leuven (BE); Youri Ponomarev, Leuven (BE); Remco Pijnenburg, Hoogeloon (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/995,846

(22) PCT Filed: Jul. 11, 2006
(86) PCT No.: PCT/IB2006/052348

§ 371 Date: Jan. 16, 2008

Related U.S. Application Data
(60) Provisional application No. 60/701,337, filed on Jul. 21, 2005.

(51) Int. Cl. A61B 5/04

(52) U.S. Cl. 600/377

(57) ABSTRACT

An apparatus and method for improving electrical contact between an implanted device (10) for recording or stimulating neuronal activity and surrounding tissue (12) (e.g., brain tissue, nerve fibers, etc.). In an exemplary embodiment, a nanometer sized topographic structure (36, 136) (e.g., a nanometer scale pillar) is processed for electrical connection with a corresponding electrode (30, 32) of the implanted device (10). The nanometer scale topographic structure (36, 136) bridges a gap (26) between the implanted device (10) and surrounding tissue (12), thus improving neuron-electrode coupling therebetween. The present disclosure can also be extended to any application where capacitive coupling to single or multiple cells (20) can be used for sensing and/or stimulation thereof.

The present disclosure relates generally to topographic structures extending from an implanted device for the electrical stimulation and/or detection of biological tissue. In particular, the present disclosure relates to nanometer scale topographic structures extending from electrodes of an implantable medical device to improve neuron-electrode coupling.

Electrodes are chronically implanted into the human body for stimulating nervous and muscular tissue. For example, such chronically implanted electrodes are used in pacemakers, for deep brain stimulation in Parkinson disease and for the functional electrical stimulations of muscles in paralysed persons. Similar chronically implanted electrodes can also record neural or muscular activity (e.g., for control of prostheses and closed loop systems for deep brain stimulation (DBS)) by recording action potentials or field potentials.

Due to the trauma inflicted during the implantation procedure of the electrode, there is usually an acute inflammatory response of the tissue surrounding the electrode. After the acute inflammation subsides after about several days or several weeks, the immune system reacts by forming an encapsulating tissue layer around the foreign body (e.g., implanted electrode). The encapsulating tissue layer prevents direct contact between the electrode and surrounding nerve tissue. The lack of direct contact is especially relevant for recording electrodes, since the signals from the nerve cells are very weak and the encapsulation layer can lead to a failure of contact between the electrode and nerve tissue after some weeks to months. Stimulating electrodes are not affected as much, because the stimulus amplitude can be increased to compensate for the decrease in coupling efficiency. However, increasing the stimulus amplitude results in increases in costs due to an increase in energy consumption and a reduction in the lifetime of an implanted battery. The reduced lifetime of the battery also means more frequent changes of the battery, thus necessitating more surgical operations to change the same. Furthermore, increasing the amplitude of the stimulus signal may reduce the spatial resolution of stimulation (larger volume is addressed, less controllable stimuli due to tissue inhomogeneity), this is especially important for applications such as a retina implant for restoration of vision or functional electrical stimulation (FES) for restoration of limb movement in paralyzed persons. A better spatial resolution also reduces side effects in applications such as DBS. Moreover, it is contemplated that many future implantable medical devices will involve recording capabilities in order to give feedback for controlling the stimulus as in a closed-loop system, thus requiring a reduction or complete suppression of the formation of an encapsulating tissue layer.

Even if the formation of the encapsulating tissue layer can be completely prevented, an electrode and subject cell are still prevented from direct physical contact with one another. Long glycoprotein chains protruding from the cell membrane (glycocalix, consists of e.g., laminin and fibronectin) act as a cushion surrounding the cell and form a cleft between the cell and electrode surface. Flourescence interference contrast microscopy (FLIC) measurements on cell cultures have revealed a gap of 50-100 nm between neurons and a SiO₂ surface (e.g., electrode substrate), depending on the coating on the substrate surface (e.g., poly-L-lysine, Laminin). This gap can be reduced to almost 0 nm only for artificial lipid vesicles (e.g., a pure lipid bilayer without glycocalix), but not for real cells.

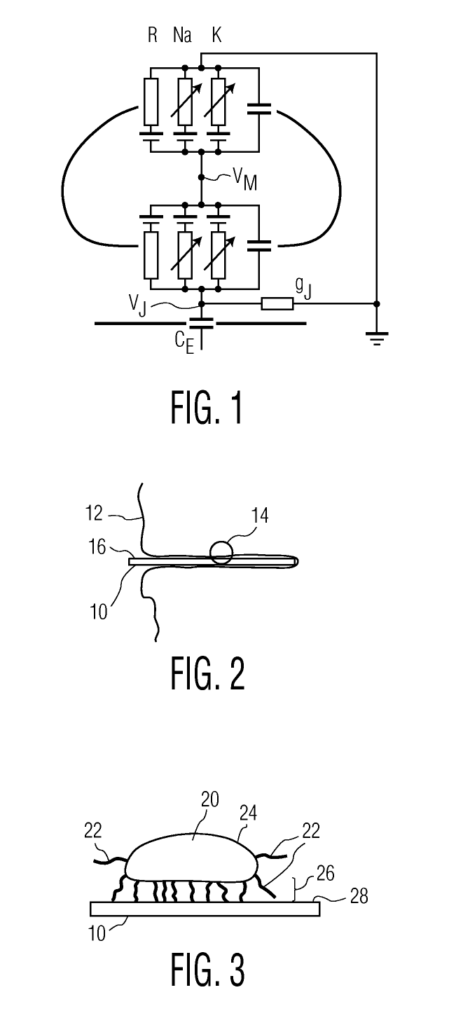

FIG. 1 illustrates an equivalent circuit for a non-invasive extracellular coupling to a capacitive electrode represented by a capacitance C_{E} (point-contact model). The neuron in FIG. 1 is represented by a Hodgkin-Huxley membrane circuit model. The cell membrane separates the interior of the cell from the extracellular liquid and acts as a capacitor. Passive and voltage-gated ion-channels are incorporated into the cell membrane allowing the passage of (specific) ions. They are represented as resistors with constant (passive channels) and variable (active channels) conductance. Because of active ion transport through the cell membrane (e.g., ion pump), the ion concentration inside the cell is different from that in the extracellular liquid. The Nernst potential generated by the difference in ion concentration is represented by a battery for every type of ion (e.g., Na, K, and leak are relevant in the Hodgkin-Huxley model). Stimulating the neuron (depolarizing stimulus above firing threshold) leads to a transient opening of voltage-gated ion-channels (governed by the channel dynamics) and a short (1 to several milliseconds) increase in membrane potential (about 100 mV) called action potential.

For metal electrodes, the capacitance C_{E} is replaced by a parallel circuit of capacitance and resistance. V_{M} is the intracellular voltage and g_{J} is the area specific conductance of a cleft between the cell and electrode surface. V_{J} is the voltage in the cleft (junction) between the electrode and cell and is connected to the grounded bath by g_{J}. The neuron is represented by parallel circuits of resistances in series with corresponding voltage sources and a capacitance according to the Hodgkin-Huxley Model. The neuron is represented with two of these circuits, one for the adherent and one for the free membrane.

For recording extracellular activity, the electrode measures V_{J}, which results from a voltage drop created by the ionic currents (released from the neuron during firing of an action potential) along the conductance gⱼ. This voltage drop increases with decreasing g_{J} meaning a larger signal at the electrode. To stimulate neural activity, for example, voltage pulses are applied to the capacitor C_{E} (or constant currents for metal electrodes) that modulate V_{J}. Again, the coupling efficiency is increased with smaller values of g_{J}, since g_{J} is the reciprocal resistance, 1/R_{J}. The conductance g_{J} is determined by the specific electrolyte resistance ρⱼ and the thickness d of the cleft according to: g_{J}=d/ρ_{J}.

According to the relation above, coupling efficiency for both stimulating and recording can be increased either by decreasing d (e.g., decreasing the width of the cleft) or increasing the specific electrolyte resistance ρ_{J}. A more detailed description of the interfacing between a neuron and a capacitive electrode can be found in P. Fromherz. Neuroelectronic Interfacing, In R. Waser, ed., Nanoelectronics and Information Technology. Wiley-VCH, Berlin (2003), 783-810 and in R. Schaetzthauer and P. Fromherz, Neuron-silicon junction with voltage-gated ionic currents, Eur. J. Neurosci. 10 (1998), 1956-1962, both of which are incorporated herein by reference in their entirety.

Thus, there is a need for an apparatus and method for increasing the electrical coupling between neurons and electrodes by locally reducing the specific conductance of the cleft between cell and electrode surface.

The present disclosure provides an apparatus for increasing electrical coupling between an electrode and a target biological cell of biological tissue. In one embodiment the apparatus includes a support structure; an array of electrodes arranged in or on the support structure; and a plurality of pillar structures extending from corresponding electrodes. The pillars are dimensioned in nanometer scale to overcome a glycocalix cushion separating the cell from the terminal end of the pillar, thus increasing electrical coupling between the electrodes and the targeted biological cell.

The present disclosure also provides a method for increasing electrical coupling between an electrode and a target biological cell of biological tissue. In one embodiment, the method includes: arranging an array of electrodes in or on a support structure; and dimensioning a plurality of pillar structures in nanometer scale to extend from corresponding electrodes. The nanometer scale pillar structures overcome a glycocalix cushion separating the cell from a terminal end defining each pillar, thus increasing electrical coupling between the electrodes and the targeted biological cell.

Additional features, functions and advantages associated with the disclosed apparatus and method will be apparent from the detailed description which follows, particularly when reviewed in conjunction with the figures appended hereto.

To assist those of ordinary skill in the art in making and using the disclosed apparatus and method, reference is made to the appended figures, wherein:

FIG. 1 is a schematic circuit view of a point-contact model describing the electrical coupling of a neural cell to a capacitive electrode in close proximity, the neuron being represented by the Hodgkin-Huxley model;

FIG. 2 is a cross sectional view of a prior art implanted electrode device in tissue;

FIG. 3 is an enlarged view of the circle of FIG. 2 illustrating a gap between a cell membrane of a neuron of the tissue and the implanted electrode device;

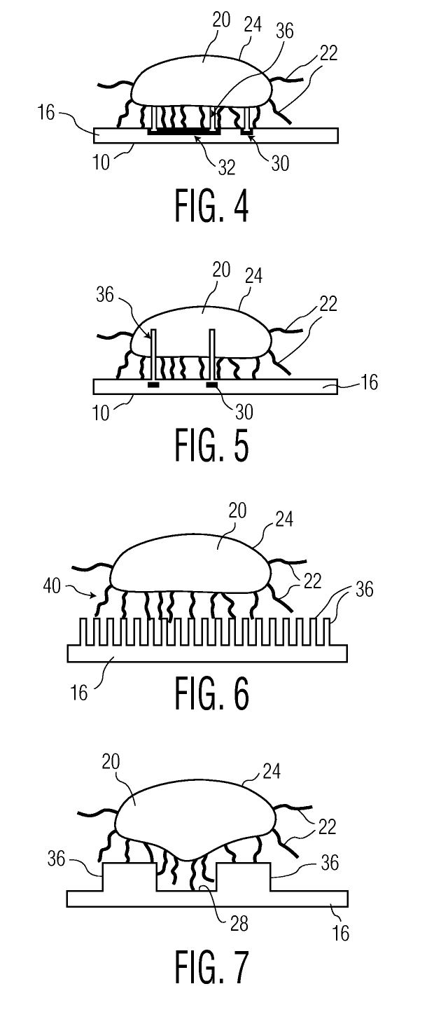

FIG. 4 is a cross sectional view of an implanted electrode device illustrating pillars extending from individual electrodes disposed on a substrate of the implanted electrode device and in electrical contact with the cell membrane in accordance with an exemplary embodiment of the present disclosure;

FIG. 5 is a cross sectional view of an implanted electrode device illustrating pillars extending from electrodes disposed on a substrate of the implanted electrode device and extending through the cell membrane in accordance with an alternative exemplary embodiment of the present disclosure;

FIG. 6 is a cross sectional view of a dense array of nm pillar structures defining a top layer of the imbedded substrate and a glycocalix cushion extending from a neuron illustrating a gap and prevention of contact with the cell membrane as a result of the dense array;

FIG. 7 is a cross sectional of view of a less dense array of larger pillar structures compared to FIG. 6 illustrating prevention of contact with the cell membrane having a glycocalix cushion extending therefrom;

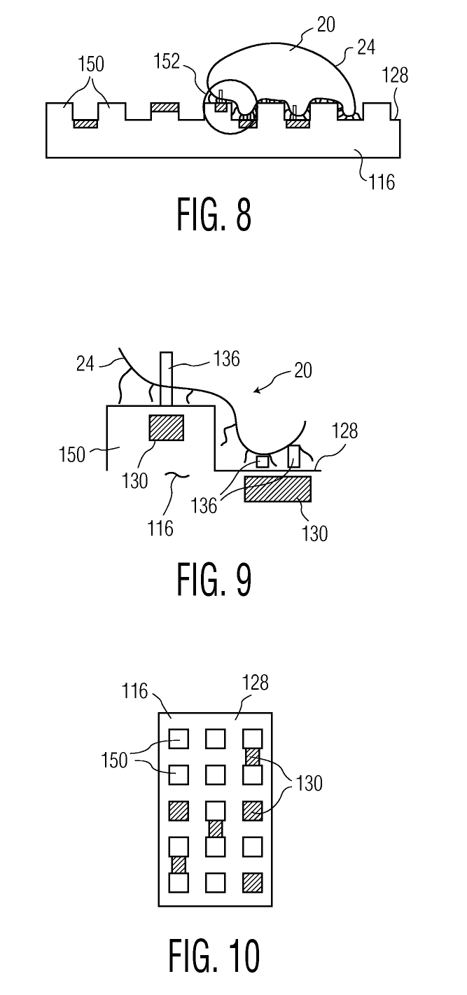

FIG. 8 is a cross sectional view of a combination of μm topographic structures defining a top layer of the electrode substrate and a glycocalix cushion extending from a neuron illustrating a plurality of nm pillar structures extending from corresponding electrodes either in abutting contact or penetrating the cell membrane of the neuron in accordance with an exemplary embodiment of the present disclosure;

FIG. 9 is an enlarged view of the circle indicated in FIG. 8; and

FIG. 10 is a top plan view of the embedded substrate of FIGS. 8 and 9 illustrating an array of μm topographic structures having an irregular distribution of electrodes in accordance with an exemplary embodiment of the present disclosure.

As set forth herein, the apparatus and method of the present disclosure advantageously increases the neuron-electrode coupling efficiency by locally reducing the cleft between a nerve cell and electrode surface. The cleft between the nerve cell and electrode surface is reduced with pillar like structures protruding from the electrode surface to permit and facilitate neural tissue interfacing, e.g., in implantable neurostimulation medical devices. The present disclosure can be extended to any application where electrical coupling to single or multiple cells is desired for either sensing or stimulation thereof. More specifically, the present disclosure suggests using pillars having a very small surface area (e.g., small diameter pillars) to avoid glycocalix molecules from attaching at a terminal end or top that would prevent direct contact between the pillar and cell membrane. In addition, the present disclosure suggests using pillars having a small overall density (e.g. less than 10 pillars beneath the contact area of a neuron with the electrode). Otherwise, the glycocalix can form a cushion on top of the pillars due to entropic effects obstructing the action of the pillars.

By using such an approach, significant improvements in spatial resolution, selectivity, signal to noise ratio, and power consumption can be achieved. Also, the apparatus of the present disclosure requires less electronics, smaller and less costly electronics and relies on mainstream IC manufacturing techniques, making it cost-effective.

With reference to FIG. 2, an electrode device 10 is illustrated implanted in biological tissue 12. FIG. 3 is an enlarged view of circle 14 in FIG. 2. FIG. 3 depicts a neural cell or neuron 20 of tissue 12 having long glycoprotein chains (glycocalix) 22 protruding from a cell membrane 24 acting as a cushion surrounding the cell 20 forming a cleft 26 between the cell 20 and an electrode surface 28 of device 10. The implant device 10 is shown inserted into tissue 12 without an encapsulation layer, while neuron 20 is separated from the implant surface by the glycocalix cushion defined by the plurality of chains of glycoproteins 22 forming cleft 26. Both FIGS. 2 and 3 illustrate device 10 as an implanted planar substrate 16 having electrodes (not shown) for electrical coupling with cell membrane 24. However, the gap or cleft 26 formed by glycocalix 22 prevents actual contact therebetween resulting in a small amplitude for any signal generated or received by the electrodes of device 10.

FIG. 4 illustrates substrate 16 having electrodes 30, 32 embedded therewith. Electrode 30 includes a single nanometer scale pillar structure 36 having one end mechanically and electrically coupled to electrode 30, while an opposite terminal end is electrically coupled to cell membrane 24 via abutting engagement therewith. Electrode 32 includes a plurality of nanometer scale pillar structures 36 each having one end mechanically and electrically coupled to electrode 32, while an opposite terminal end is electrically coupled to cell membrane 24 via abutting engagement therewith. Each of the nanometer scale pillar structures 36 are long enough to close a gap created by glycocalix 22 between the cell membrane 24 and electrode surface 28 to improve neuron-electrode coupling therebetween.

In exemplary embodiments, a diameter of each pillar structure is less than about 50 nm, wherein the lower limit is defined by the mechanical stability of the structure 36. A height of each of the pillar structures 36 as illustrated in FIG. 4 is between about 50 nm to about 100 nm wherein the pillar structures 36 are in abutting contact with the cell membrane 24.

FIG. 5 illustrates substrate 16 having a pair of electrodes 30 embedded therewith. Each electrode 30 includes a single nanometer scale pillar structure 36 having one end mechanically and electrically coupled to electrode 30, while an opposite terminal end penetrates neuron 20. FIG. 5 illustrates a second configuration in which the exposed terminal ends of each pillar structure 36 penetrate the cell membrane and extend into the intracellular space defining the cell 20. A height of each of the pillar structures of FIG. 5 is between about 100 nm and about 300 nm. In this manner, structures 36 of FIG. 5 provide invasive contact with cell 20, while structures 36 of FIG. 4 provide non-invasive contact with cell 20. However, the cell membrane 24 of cell 20 of FIG. 5 may not rupture and adhere well to the surface of structure 36 if it does not move providing a very stable configuration.

In both the exemplary embodiments depicted in FIGS. 4 and 5, the aspect ratios for both configurations of pillar structures is greater than 2. In other words, the length or height to diameter ratio is greater than 2.

The pillar structures 36 may be fabricated of a metal or other conducting material. In other embodiments, the pillar structures include a conductive core covered with a dielectric, similar to capacitive electrodes.

The pillar structures 36 may be connected to the electrodes either individually or in small groups (e.g., 2-3 pillars). In any case, there should be few (e.g., less than 10) pillar structures per electrode to maintain an overall small density and to prevent glycocalix from forming a cushion 40 on a dense array of pillar structures 36 (as shown in FIG. 6). For example, the electrodes 30, 32 may be embodied as metal pads, as illustrated in FIGS. 4 and 5 or transistors, such that the pillar structures themselves are the electrodes, or a major part of the electrode.

Furthermore, it should be noted that the pillar structures 36 are rigid to allow penetration of the cell membrane 24, whereas a pillar structure 36 formed of a flexible polymer or single polymer chains used as pillars, for example, would not facilitate such penetration of the cell membrane. In addition, the structures 36 of the present disclosure may be deposited at well-defined locations and at defined ‘concentrations’, allowing single pillar structures 36 to be connected to electronic circuits. As discussed above, the pillar structures may be formed either as metal/conducting structures or conductive structures with a dielectric surface for capacitive coupling (the latter preventing faradic currents across the electrode-electrolyte interface).

The high aspect ratio pillar structures described by the present disclosure can be processed onto planar substrates 16 by standard processing techniques, including masking and anisotropic etching, for example, which can then be followed by further isotropic etches to further thin down the structures 36. Alternatively, the pillar structures can also be fabricated by selective growth techniques (e.g., similar to the growth of nanowires).

FIG. 6 illustrates a plurality of pillar structures 36 each having a suitable aspect ratio, but arranged in an array that is too dense. In this situation, glycocalix 22 forms a cushion 40 between cell membrane 24 and surface 28 of substrate 16 preventing contact with the cell membrane. Thus, cushion 40 prohibits effective coupling therebetween resulting in a low signal to noise ratio for any signals between electrodes operably connected to the pillar structures 36 and cell membrane 24.

FIG. 7 illustrates a pair of pillar structures 36 each having a low aspect ratio or structures having a much too large of a diameter. In this situation, glycocalix 22 forms a cushion 40 between cell membrane 24 and surface 28 of substrate 16 preventing contact with the cell membrane, as in the dense array of FIG. 6. Thus, cushion 40 prohibits effective coupling therebetween resulting in a low signal to noise ratio for any signals between electrodes operably connected to the pillar structures 36 and cell membrane 24.

FIGS. 8-10 illustrate a non-planar substrate 116 having an array of μm square posts 150 extending from a surface 128 defining substrate 116. In this manner, substrate 116 with posts 150 extending therefrom define a three dimensional (3-D) topographic structure surface having electrodes 130 disposed in an irregular pattern as best seen with reference to FIG. 10. FIGS. 8-10 illustrate electrodes 130 located at the top of posts 150 and intermediate adjacent posts 150 on surface 128 of substrate 116. As best seen in FIG. 9, pillar structures 136 can be disposed at a top of square posts 150 and/or at surface 128 for electrical coupling with cell membrane 24. FIG. 9 is an enlarged view of circle 152 in FIG. 8. As illustrated in FIG. 9, one of the pillar structures 136 penetrates the cell membrane 24 and extends into the intracellular portion of cell 20, while the remaining structure 136 abuts the cell membrane without penetrating therethrough.

It should be noted that although posts 150 are described as square posts, the present disclosure is not limited thereto, as other geometries are contemplated, including circular and elliptical columns, for example. Further, a regular distribution of electrodes 130 is also contemplated and is not limited to the irregular distribution illustrated. In principle, the electrodes 130 may also be arranged at the vertical walls defining the topographic posts 150 extending from surface 128. The 3-D topographic structured surface can be processed into planar substrates by standard processing techniques (e.g., masking and etch). Alternatively, the 3-D topographic structured surface could also be made by embossing or injection moulding of suitable polymers.

The primary focus of the present disclosure is not suppressing the formation of an encapsulating tissue layer, but to directly contact the cell by overcoming the glycocalix cushion separating the cells from the electrodes surfaces using conductive structures of extremely small density and having a high aspect ratio. However, it will be recognized by those skilled in the pertinent art that this is only possible if there is no encapsulating tissue layer. It has been shown with respect to in vitro experiments (e.g., cell cultures) that topographic morphologies such as that shown in FIGS. 8-10 can affect cell morphology and growth, which could also reduce or completely prevent formation of an encapsulating tissue layer that usually forms around implanted electrodes. The μm-scale 3D patterning of electrode surfaces such as that disclosed in FIGS. 8-10 might be suitable to suppress the growth of scar tissue and glia cells and to promote the growth of neural cells.

The topographic structures described in the present disclosure can be applied to all implantable medical electrodes especially for devices where high spatial resolution and low power consumption is desirable such as retina implants, deep brain stimulation (DBS) electrodes, electrodes for recording (e.g., motorcortex and control of prostheses) and stimulating brain activity (e.g., somatosensory cortex or deliver sensory input from a camera).

In one aspect of the invention, a neural modulation system for use in treating disease which provides stimulus intensity which may be varied is disclosed. The stimulation may be at least one of activating, inhibitory, and a combination of activating and inhibitory and the disease is at least one of neurologic and psychiatric. For example, the neurologic disease may include Parkinson's disease, Huntington's disease, Parkinsonism, rigidity, hemiballism, choreoathetosis, dystonia, akinesia, bradykinesia, hyperkinesia, other movement disorder, epilepsy, or the seizure disorder. The psychiatric disease may include, for example, depression, bipolar disorder, other affective disorder, anxiety, phobia, schizophrenia, multiple personality disorder. The psychiatric disorder may also include substance abuse, attention deficit hyperactivity disorder, impaired control of aggression, or impaired control of sexual behavior.

In another aspect of the invention, a neurological control system is disclosed. The neurological control system modulates the activity of at least one nervous system component, and includes at least one stimulating electrode, each constructed and arranged to deliver a neural modulation signal to at least one nervous system component; at least one sensor, each constructed and arranged to sense at least one parameter, including but not limited to physiologic values and neural signals, which is indicative of at least one of disease state, magnitude of symptoms, and response to therapy; and a stimulating and recording unit constructed and arranged to generate the neural modulation signal based upon a neural response sensed by the at least one sensor in response to a previously delivered neural modulation signal.

The disclosed apparatus and method optimizes the efficiency of energy used in the treatment given to the patient by minimizing to a satisfactory level the stimulation intensity to provide the level of treatment magnitude necessary to control disease symptoms without extending additional energy delivering unnecessary overtreatment and wasting energy, as well as to minimize side effects. In present stimulation systems, a constant level of stimulation is delivered over a large area, resulting in either of two undesirable scenarios when disease state and symptoms fluctuate: (1) undertreatment, i.e. tremor amplitude exceeds desirable level, or (2) overtreatment or excess stimulation, in which more electrical energy is delivered than is actually needed. In the overtreatment case, battery life is unnecessarily reduced. The energy delivered to the tissue in the form of a stimulation signal represents a substantial portion of the energy consumed by the implanted device; minimization of this energy substantially extends battery life, with a consequent extension of time in between reoperations to replace expended batteries. Furthermore, by optimizing the coupling efficiency side effects can be reduced because stimulation is well localized to the target tissue and other tissue remains unaffected. In addition, the apparatus of the present disclosure relies on mainstream IC manufacturing techniques providing a cost effective solution to the prior art.

The disclosed method and apparatus increase the signal to noise ratio for recording neuronal activity with extracellular recording devices. This means that less complex signal processing for action potential detection is required, including less electronics required, less power consumption, smaller and cheaper devices. Further, activity from adjacent neurons can be discriminated allowing a high spatial resolution of recordings.

The amplitude for triggering action potentials can be reduced resulting in a decreased power consumption and increased lifetime of the implant's battery. Furthermore, due to their reduced amplitude, stimuli from close-by electrodes do not overlap any more enabling also higher spatial resolution for stimulation.

Although the method and apparatus of the present disclosure has been described with reference to exemplary embodiments thereof, the present disclosure is not limited to such exemplary embodiments. Rather, the apparatus disclosed herein is susceptible to a variety of modifications, enhancements and/or variations, without departing from the spirit or scope hereof. Accordingly, the present disclosure embodies and encompasses such modifications, enhancements and/or variations within the scope of the claims appended hereto.

1. An apparatus for increasing electrical coupling between an electrode (30, 32) and a target biological cell (20) of biological tissue (12), the apparatus comprising:
a support structure (16);
an array of electrodes (30, 32) arranged in or on the support structure (16); and
a plurality of pillar structures (36, 136) extending from corresponding electrodes (30, 32), the pillars (36, 136) being dimensioned in nanometer scale to overcome a glycocalix cushion (26, 40) separating the cell (20) from the terminal end of the pillar (36, 136) thus increasing electrical coupling between the electrodes (30, 32) and the targeted biological cell (20).

2. The apparatus of claim 1, wherein the array of electrodes (30, 32) includes at least one of a sensing electrode and a stimulation electrode.

3. The apparatus of claim 1, wherein a density of the pillars (36, 136) in contact with the biological cell (20) is less than 10 pillars (36, 136) per electrode (30, 32).

4. The apparatus of claim 1, wherein a diameter of each pillar (36, 136) is less than about 50 nm.

5. The apparatus of claim 1, wherein a length of each pillar (36, 136) having a terminal end abutting a cell membrane (24) of the biological cell (20) is between about 50 nm and about 100 nm.

6. The apparatus of claim 1, wherein a length of each pillar (36, 136) having a terminal end penetrating a cell membrane (24) and into intracellular space of the biological cell (20) is between about 100 nm and 300 nm.

7. The apparatus of claim 1, wherein each pillar (36, 136) is made of a metal or other conducting material.

8. The apparatus of claim 1, wherein each pillar (36, 136) includes a conducting core covered by a dielectric.

9. The apparatus of claim 1, wherein the support structure (16) includes a 3-D topographic structure (150), each topographic structure (150) having dimensions of about 1 nm to about 20 μm.

10. The apparatus of claim 9, wherein the 3-D topographic structure (150) includes a shape of one of round, elliptic, square, rectangular, triangular, and quadratic, the structure (150) preventing formation of an encapsulating tissue layer around the electrode device (30, 32).

11. The apparatus of claim 9, wherein the 3-D topographic structure (150) is formed by one of processing into planar substrates using one of standard semiconductor processing methodology, embossing of suitable polymers and injection molding of suitable polymers.

12. The apparatus of claim 1, wherein the cell (20) is a neural cell.

13. The apparatus of claim 12, wherein at least a portion of the electrodes (30, 32) are used for electrical coupling to at least one of record an action potential and stimulate generation of an action potential or block a propagation of an action potential along an axon of the neural cell (20).

14. The apparatus of claim 1, wherein at least a portion of the electrodes (30, 32) are stimulation electrodes (30, 32) providing stimulation to the biological tissue (12) for at least one of activating, inhibitory, and a combination of activating and inhibitory.

15. A method for increasing electrical coupling between an electrode (30, 32) and a target biological cell (20) of biological tissue (12), the method comprising:
arranging an array of electrodes (30, 32) in or on a support structure (16); and
dimensioning a plurality of pillar structures (36, 136) in nanometer scale to extend from corresponding electrodes (30, 32), the nanometer scale pillar structures (36, 136) overcoming a glycocalix cushion (26, 40) separating the cell (20) from a terminal end defining each pillar (36, 136) thus increasing electrical coupling between the electrodes (30, 32) and the targeted biological cell (20).

16. The method of claim 15, wherein a density of the pillars (36, 136) in contact with the biological cell (20) is less than 10 pillars per electrode (30, 32).

17. The method of claim 15, further comprising dimensioning a diameter of each pillar (36, 136) less than about 50 nm.

18. The method of claim 15, further comprising dimensioning a length of each pillar (36, 136) having a terminal end abutting a cell membrane (24) of the biological cell (20) between about 50 nm and about 100 nm.

19. The method of claim 15, further comprising dimensioning a length of each pillar (36, 136) having a terminal end penetrating a cell membrane (24) and into intracellular space of the biological cell (20) between about 100 nm and 300 nm.

20. The method of claim 15, further comprising fabricating each pillar (36) of one of a metal or other conducting material and a conductive core covered with a dielectric.
